(19) United States
(12) Patent Application Publication
(10) Pub. No.: US 2022/0383979 A1
(43) Pub. Date: Dec. 1, 2022

(54) NUCLEIC ACID MASS SPECTRUM NUMERICAL PROCESSING METHOD

(71) Applicant: ZHEJIANG DIGENA DIAGNOSTIC TECHNOLOGY CO., LTD., Hangzhou City, Zhejiang (CN)

(72) Inventors: Jianwei SHU, Hangzhou City, Zhejiang (CN); Shuanghong XIANG, Hangzhou City, Zhejiang (CN); Songjiong WANG, Hangzhou City, Zhejiang (CN)

(21) Appl. No.: 17/771,216

(22) PCT Filed: Dec. 9, 2020
(86) PCT No.: PCT/CN2020/134810

§ 371 Date: Apr. 22, 2022

(30) Foreign Application Priority Data
Feb. 10, 2020 (CN) 202010084107.4

(51) Int. Cl. G16B 30/00; G06K 9/00; H01J 49/00

(52) U.S. Cl. CPC G16B 30/00; G06K 9/0053; G06K 9/00516; H01J 49/0036

(57) ABSTRACT

A numerical processing method for a nucleic acid mass spectrum, including: step S1, recalibrating a single mass spectrum, for each detection point of a sample, obtaining a plurality of mass spectra corresponding to different positions of the detection point, each mass spectrum being recalibrated by using anchor peaks with an expected mass-to-charge ratio; step S2, synthesizing the mass spectra, where the mass spectra corresponding to the different positions of the detection point are synthesized into a unitary mass spectrum of the detection point; step S3: performing wavelet filtering on the unitary mass spectrum to eliminate high-frequency noise and a baseline through a wavelet-based digital filter; and step S4: extracting a peak feature value, performing peak fitting to obtain a fitted curve of the unitary mass spectrum, and obtaining a peak height, a peak width, a peak area, a mass offset, and a signal-noise ratio based on the fitted curve.

### TECHNICAL FIELD

The present disclosure belongs to a technical field of nucleic acid mass spectrum, and particularly relates to a numerical processing method for a nucleic acid mass spectrum.

### BACKGROUND

Mass spectrum technology has been widely used in biological analysis in recent years because of the advantages of rapidity, accuracy, and high sensitivity. As the basic material of life, nucleic acid plays a vital role in the major life phenomena, such as growth, development, reproduction, inheritance, and variation, of organisms. Modern biotechnology has found that most of the physiological or disease traits are expressed by a series of gene regulation existing in the nucleic acid sequence. Therefore, for the nucleic acid, accurate nucleotide detection is particularly important. As an indispensable part before the nucleotide detection, numerical processing of a mass spectrum is of self-evident importance. At present, there are problems such as low conversion rate of the acquired data and uneven data in similar methods, these problems seriously affect the result of the nucleotide detection, and the further research is needed in this regard.

### SUMMARY

The purpose of the present disclosure is to solve the problems of low conversion rate, uneven data, and the like in the mass spectrum data acquisition process, and the present disclosure provides a numerical processing method for a nucleic acid mass spectrum to extract reliable feature values before gene analysis, and the numerical processing method is a numerical processing method for a nucleic acid mass spectrum aiming at ameliorating the limitation of the prior art and improving the accuracy of nucleotide detection.

The present invention is achieved through the following technical solutions:

A numerical processing method for a nucleic acid mass spectrum, comprising following steps:

step S1, recalibrating a single mass spectrum, for each detection point of a sample, obtaining a plurality of mass spectra corresponding to different positions of the detection point, where each mass spectrum needs to be recalibrated by using a group of special peaks, namely anchor peaks, with an expected mass-to-charge ratio;
step S2, synthesizing the plurality of mass spectra, where on a basis of the step S1, the plurality of mass spectra corresponding to the different positions of the detection point are synthesized into a unitary mass spectrum of the detection point;
step S3: performing wavelet filtering, on a basis of the step S2, to eliminate high-frequency noise and a baseline through a wavelet-based digital filter; and
step S4: extracting a peak feature value, performing peak fitting on a basis of the step S3, and obtaining a peak height, a peak width, a peak area, a mass offset, and a signal-noise ratio based on a fitted curve of the unitary mass spectrum of the detection point.

As a preferred embodiment, in the step S1, recalibrating the single mass spectrum comprises:

step S11: selecting a candidate reference peak, and selecting a group of reference peaks from all possible expected peaks according to following criteria: 1, a peak value of a reference peak being within a mass range of a specific interval; 2, no reference peak, adjacent to the reference peak, existing in the mass range of the specific interval;
step S12, positioning a peak, and applying a weight matrix convolution filter with a width of 9 to the mass spectrum, where the weight matrix convolution filter is preferably: (−4, 0, 1, 2, 2, 2, 1, 0, −4), for a given point of the mass spectrum, an intensity value of the given point after applying the weight matrix convolution filter is equal to a weighted sum of 9 values around the given point, and is expressed by a following formula:

$${y}_{i}^{′} = {\sum }_{k = 1}^{9} {I}_{k} {y}_{k} + i$$

decomposing the total mass spectrum into a plurality of specific point intervals based on filtered intensity values, identifying a local noise for each interval, and identifying a peak with an intensity greater than or equal to four times the local noise and greater than or equal to a global minimum value as a candidate peak, where the global minimum value is: 0.01*a maximum local maximum value;
step S13: fitting a peak of the mass spectrum;
step S14: finally selecting an anchor peak, for a detected peak list, finding a cut-off SNR (i.e., a minimum SNR), matching a peak in the detected peak list with a list of candidate reference peaks, and only selecting a peak whose mass is within a specific range of the candidate reference peak and whose SNR is higher than the cut-off SNR;
step S15: performing a recalibration operation, calculating a calibration coefficient by a nonlinear fitting method in combination with the anchor peak obtained and an expected mass of the anchor peak, where it is assumed that a mapping function between a mass spectrometer and m/z (mass-to-charge ratio) is a Bruker function in a form of m=A(\sqrt{Bt+C}−1)².

Further, the specific steps of fitting a peak in the step S13 comprises:

step S131: determining an expected line width;
step S132: masking a region of an expected signal within an interval of NN expected line widths, NN being preferably 4;
step S133: calculating an average of an intensity yᵢ of the mass spectrum within a MMλₘ interval as an implicit baseline, where λₘ is a smallest estimated line width in the interval, MM is preferably 80, and in a masked region of the interval, a value of the intensity yᵢ is provided by linear interpolation;
step S134: calculating an effective value (Root Mean Square, RMS) of a (signal-baseline) operation as a noise level;
step S135: masking a point, and further masking a point, having a SNR(SNR calculated as a ratio of the peak height to a noise) greater than a given value and a noise greater than a given value, in a peak region;
step S136, determining a region having specific estimated line widths of each peak as a fitted region, and in a case of no overlapping peaks, fitting a single Gaussian peak by Levenberg-Marquardt algorithm to find specified parameters to minimize a tuning function.

Further, in the step S131, the specific step to determine the expected line width comprises:

λₑ=L_{A}+L_{B}·M

where L_{A} and L_{B} are default parameters, and M is a given peak value (Da).

Further, in the step S136, the specific step for the tuning function comprises:

$${χ}^{2} = {Σ [ \frac{{y}_{i} - {H}_{f} \cdot exp [ - {( \frac{{M}_{f} - {m}_{i}}{{λ}_{f}} )}^{2} ]}{{σ}_{i}} ]}^{2}$$

where a sum is obtained by summing all {yᵢ, mᵢ} from a specified interval, H_{f} is a fitting height above the baseline corresponding to a point M_{f}; a parameter M_{f} represents a fitting mass, a parameter λ_{f} represents a fitting line width, and σᵢ is a certain parameter according to a given condition.

As another preferred embodiment, the specific steps of extracting the peak feature value in the step S4 include:

step S41: fitting a peak of the mass spectrum, the same as the step S13;
step S42: recording following features:
1, a height above a baseline of a center of a fitted peak, H_{f},
2, a fitting line width, λ_{f},
3, a peak offset (a distance between the center of the fitted peak and a center of an expected peak), δ_{f}=M_{f}−Mₑ,
4, an area A of a region between the fitted peak and the baseline within a range of 4λ_{f},
5, a signal-noise ratio, SNR=H_{f}|N(M_{f}),
6, an area variance, V=A/SNR
7, a fitting area difference Δ, a square root of a sum of a square difference between a fitted intensity and a measured intensity.

The present disclosure has the following advantages: 1. the numerical processing method for the nucleic acid mass spectrum provided by the present disclosure can extract reliable feature values before the gene analysis, and is a numerical processing method for a nucleic acid mass spectrum aiming at ameliorating the limitation of the prior art and improving the accuracy of nucleotide detection; 2. the numerical processing method for the nucleic acid mass spectrum can improve the credibility of nucleic acid mass spectrum data acquisition.

### BRIEF DESCRIPTION OF THE DRAWINGS

FIG. 1: mass spectrum before filtering;

FIG. 2: mass spectrum after filtering;

FIG. 3: comparison diagram before and after peak fitting.

### DETAILED DESCRIPTION

The content of the present disclosure is further described below with reference to the accompanying drawing and embodiments.

The present disclosure relates to a numerical processing method for a nucleic acid mass spectrum, and the numerical processing method comprises the following steps:

step S1: recalibrating a single mass spectrum. For a sample, a plurality of mass spectra (e.g., n mass spectra, and typically n=5) corresponding to different positions of a detection point of the sample. Each mass spectrum is actually a sum of the mass spectra obtained by a plurality of times of laser excitation (e.g., m times, and typically m=20). An initial coefficient of the mass spectrum is generated based on the assumption that the mapping function between the mass spectrometer and m/z (mass-to-charge ratio) is a quadratic function (a form of the function is: m=At²+Bt+C). Before summing the mass spectra, the recalibration of the mass spectra is needed. The process of the recalibration is accomplished by matching a group of special identified peaks (called anchor peaks) to the expected mass thereof and follows the following steps:

In step S11: selecting a candidate reference peak, and selecting a group of clean reference peaks from all possible expected peaks according to the following criteria:

1. A peak value of a reference peak must be within a mass range of 4000 Da to 9000 Da.

2. The peak value has no adjacent reference peak within the mass range defined by the mass +/− resolution.

In step S12, positioning a peak, and applying a weight matrix convolution filter with a width of 9 to the mass spectrum. The matrix is preferably: (−4, 0, 1, 2, 2, 2, 1, 0, −4). For a given point of the mass spectrum, an intensity value of the given point after applying the weight matrix convolution filter is equal to a weighted sum of 9 values around the given point, and is expressed by a following formula:

$${y}_{i}^{′} = {\sum }_{k = 1}^{9} {I}_{k} {y}_{k} + i$$

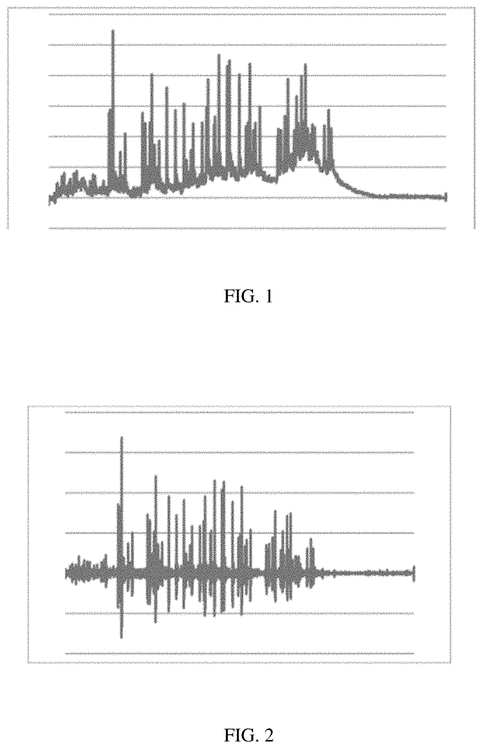

Based on the filtered intensity values, a small sliding window (n=+/−3) is used to identify a local maximum. Then, the whole mass spectrum is divided into a plurality of intervals and each interval has 500 points. For each interval, the local noise is identified as 33% of the local maximum within the surrounding window having 1500 points (+/− an interval). A peak with an intensity greater than or equal to four times the local noise and greater than or equal to a global minimum value is identified as a candidate peak, the global minimum value is preferably 0.01 times a maximum local maximum value (namely, 0.01*the maximum local maximum value). For an identified peak list, a peak is removed, where a reference peak adjacent to the peak exists within a certain range, and the peak has a SNR (ratio of the filtered intensity value to the local noise)≤2 and has a mass value outside the range of the pre-specified candidate reference peaks. Finally, the peak value index is adjusted based on the original intensity. The mass spectrum before and after the application of the filter may be referred to the FIG. 1 and FIG. 2.

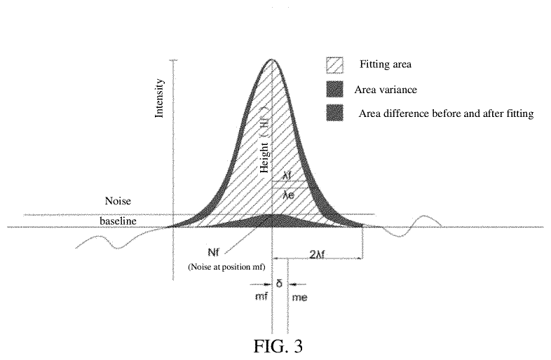

In the step S13, fitting a peak of the mass spectrum, as shown in FIG. 3, the specific implementation steps are as follows:

Step S131: determining an expected line width. The expected line width is determined by using the following formula:

λₑ=L_{A}+L_{B}·M, where L_{A} and L_{B} are the default parameters (the default value of L_{A} is 2.5, and the default value of L_{B} is 0.0005), and M is the given peak value (Da).

Step S132: masking a region of an expected signal within an interval of NN expected line widths, NN preferably being 4.

Step S133: calculating an average of an intensity yᵢ of the mass spectrum within a MMλₘ interval as an implicit baseline, where λₘ is the smallest estimated line width in this MMλₘ interval, and MM is preferably 80, and in the masked region of this MMλₘ interval, the intensity yᵢ is provided by linear interpolation.

Step S134: calculating the effective value (Root Mean Square, RMS) of the (signal-baseline) operation as the noise level.

Step S135: further masking a point, having a SNR(SNR calculated as the ratio of the peak height to the noise) great than 5 and a noise greater than 1, in the peak region.

Step S136: determining a region within four estimated line widths of each peak as a fitted region, and in a case of no overlapping peaks, fitting a single Gaussian peak by Levenberg-Marquardt algorithm to find parameters M_{f} (fitting mass) and λ_{f} (fitting line width), so that the tuning function (prototype of the function is shown below) is minimized

$${χ}^{2} = {Σ [ \frac{{y}_{i} - {H}_{f} \cdot exp [ - {( \frac{{M}_{f} - {m}_{i}}{{λ}_{f}} )}^{2} ]}{{σ}_{i}} ]}^{2}$$

The sum is obtained by summing all {yᵢ, mᵢ} from the specified interval, H_{f} is the fitting height above the baseline corresponding to the point M_{f}. For a point in the region with a distance from the peak center within 0.5λₑ, σᵢ of the point is set equal to 1, and for a point in the region with a distance from the peak center beyond 0.5λₑ, σᵢ of the point is set to 0.2 or 0.4.

Step S14: finally selecting an anchor peak, for a detected peak list, finding a cut-off SNR (i.e., a minimum SNR), matching a peak in the detected peak list with a list of candidate reference peaks, and only selecting a peak whose mass is within a +/−25 Da of the candidate reference peak and whose SNR is higher than the cut-off SNR.

Step S15: performing a recalibration, calculating a calibration coefficient by a nonlinear fitting method in combination with the anchor peak obtained and an expected mass of the anchor peak. Here, it is assumed that a mapping function between a mass spectrometer and m/z (mass-to-charge ratio) is a Bruker function in a form of m=A(\sqrt{Bt+C}−1)².

In the step S2, synthesizing the plurality of mass spectra, that is to say, on a basis of the step S1, the plurality of mass spectra corresponding to different positions of the detection point are synthesized into one mass spectrum of the detection point. The method of synthesizing the plurality of mass spectra is a “self-weighted average” method that can be described by using the following equation:

$$\overline{{I}_{i}} = \frac{{\sum }_{j = 1}^{n} {I}_{ij}^{2}}{{\sum }_{j = 1}^{n} \vert {I}_{ij} \vert}$$

where n is a count of the plurality of mass spectra, Īᵢ is the average intensity of mass i; Iᵢⱼ is an intensity of the mass i from a j-th mass spectrum.

When the mass spectra have different calibration coefficients, the optimal mass spectrum with the most anchor peaks is selected from the mass spectra. The summed mass spectrum (that is, the mass spectrum obtained by performing the “self-weighted average” method on the plurality of mass spectra) is initialized with the optimal mass spectrum. Only when the calibration coefficient of a mass spectrum and the calibration coefficient of the optimal mass spectrum meet the condition (A should change within 1%; B should change within 10%; C should change within 20 Da), the absolute intensity or the square intensity of the mass spectrum can be summed with the absolute intensity or the square intensity of another mass spectrum.

Step S3, performing wavelet filtering, the wavelet filtering being performed on the synthesized mass spectrum to eliminate the high-frequency noise and the baseline, and then, performing another round of recalibration on the filtered mass spectrum. After this round of recalibration, assigning a new coefficient A, a new coefficient B, a new coefficient C to the synthesized mass spectrum and adjusting the m/z (mass-to-charge ratio) value accordingly.

Step S4, extracting a peak feature value, as shown in FIG. 3, and the fitting process follows the following steps:
step S41: fitting a peak, the same as the step S13;
step S42: recording following features after the fitting is successful:

1, a height above a baseline of a center of a fitted peak, H_{f},

2, a fitting line width λ_{f},

3, a peak offset (a distance between the center of the fitted peak and a center of an expected peak), δ_{f}=M_{f}−Mₑ,

4, an area A of a region between the fitted peak and the baseline within a range of 4λ_{f},

5, a signal-noise ratio, SNR=H_{f}/N(M_{f}),

6, an area variance, V=A/SNR

7, a fitting area difference Δ, a square root of a sum of a square difference between a fitted intensity and a measured intensity

The numerical processing method for the nucleic acid mass spectrum according to the present disclosure can extract the reliable feature values before gene analysis, and is a numerical processing method for a nucleic acid mass spectrum aiming at ameliorating the limitation of the prior art and improving the accuracy of nucleotide detection; in addition, the method can improve the reliability of the acquired nucleic acid mass spectrum data.

1. A numerical processing method for a nucleic acid mass spectrum, comprising:
step S1, recalibrating a single mass spectrum, for each detection point of a sample, obtaining a plurality of mass spectra corresponding to different positions of the detection point, wherein each mass spectrum of the plurality of mass spectra is recalibrated by using a group of anchor peaks with an expected mass-to-charge ratio;
step S2, synthesizing the plurality of mass spectra, wherein on a basis of the step S1, the plurality of mass spectra corresponding to the different positions of the detection point are synthesized into a unitary mass spectrum of the detection point;
step S3: performing wavelet filtering on the unitary mass spectrum of the detection point, on a basis of the step S2, to eliminate high-frequency noise and a baseline through a wavelet-based digital filter; and
step S4: extracting a peak feature value, performing peak fitting to obtain a fitted curve of the unitary mass spectrum of the detection point on a basis of the step S3, and obtaining a peak height, a peak width, a peak area, a mass offset, and a signal-noise ratio based on the fitted curve of the unitary mass spectrum of the detection point.

2. The numerical processing method for the nucleic acid mass spectrum according to claim 1, wherein in the step S1, recalibrating the single mass spectrum comprises:
step S11: selecting a group of reference peaks, wherein selecting a group of reference peaks comprises selecting the group of reference peaks from all possible expected peaks according to following criteria:
1, a peak value of a reference peak being within a mass range of a specific interval;
2, no reference peak, adjacent to the reference peak, existing in the mass range of the specific interval;
step S12, positioning a peak, and applying a weight matrix convolution filter with a width of 9 to the single mass spectrum, wherein the weight matrix convolution filter is: (−4, 0, 1, 2, 2, 2, 1, 0, −4), for a given point of the single mass spectrum, an intensity value of the given point after applying the weight matrix convolution filter is equal to a weighted sum of 9 values around the given point, and is expressed by a following formula:
$${y}_{i}^{′} = {\sum }_{k = 1}^{9} {I}_{k} {y}_{k} + i$$
decomposing the single mass spectrum into a plurality of specific point intervals based on filtered intensity values, identifying a local noise for each specific point interval, and identifying a peak with an intensity greater than or equal to four times the local noise and greater than or equal to a global minimum value as a reference peak, wherein the global minimum value is: 0.01*a maximum local maximum value;
step S13: fitting a peak of the single mass spectrum;
step S14: finally selecting an anchor peak, for a detected peak list, finding a cut-off signal-noise ratio, matching a peak in the detected peak list with a list of the reference peaks, and only selecting a peak whose mass is within a specific range of the reference peak and whose signal-noise ratio is higher than the cut-off signal-noise ratio;
step S15: performing a recalibration operation, calculating a calibration coefficient by a nonlinear fitting method in combination with the anchor peak and an expected mass of the anchor peak, wherein a mapping function between a mass spectrometer and mass-to-charge ratio is a Bruker function in a form of m=A(\sqrt{Bt+C}−1)².

3. The numerical processing method for the nucleic acid mass spectrum according to claim 1, wherein the step S2 comprises:
synthesizing the plurality of mass spectra by using a self-weighted average method,
selecting an optimal mass spectrum with the most anchor peaks from the plurality of mass spectra in a case where the plurality of mass spectra have different calibration coefficients;
initializing a mass spectrum synthesized by performing the self-weighted average method on the plurality of mass spectra with the optimal mass spectrum;
summing an absolute intensity or a square intensity of a mass spectrum with an absolute intensity or a square intensity of another mass spectrum only in a case where a calibration coefficient of the mass spectrum and a calibration coefficient of the optimal spectrum meet a condition.

4. The numerical processing method for the nucleic acid mass spectrum according to claim 1, wherein, the step S3 comprises:
performing wavelet filtering on the unitary mass spectrum of the detection point to eliminate the high-frequency noise and the baseline to obtain a filtered mass spectrum, and then performing another round of recalibration on the filtered mass spectrum and adjusting a mass-to-charge ratio value accordingly.

5. The numerical processing method for the nucleic acid mass spectrum according to claim 1, wherein the step S4 comprises:
step S41: fitting a peak of the unitary mass spectrum to obtain a fitted peak;
step S42: recording following features:
1, a height above a baseline of a center of the fitted peak, H_{f},
2, a fitting line width, λ_{f},
3, a peak offset, which is a distance between the center of the fitted peak and a center of an expected peak, δ_{f}=M_{f}−Mₑ,
4, an area A of a region between the fitted peak and the baseline within a range of 4λ_{f},
5, a signal-noise ratio, SNR=H_{f}/N(M_{f}),
6, an area variance, V=A/SNR,
7, a fitting area difference Δ, a square root of a sum of a square difference between a fitted intensity and a measured intensity.

6. The numerical processing method for the nucleic acid mass spectrum according to claim 2, wherein in the step S13, steps of fitting a peak comprises:
step S131: determining an expected line width;
step S132: masking a region of an expected signal within an interval of NN expected line widths, NN being 4;
step S133: calculating an average of an intensity yᵢ of the single mass spectrum within a MMλm interval as an implicit baseline, wherein λm is a smallest estimated line width in the MMλm interval, MM is 80, and in a masked region of the MMλm interval, a value of the intensity yᵢ is provided by linear interpolation;
step S134: calculating an effective value of a signal-baseline operation as a noise level;
step S135: masking a point, and further masking a point, having a signal-noise ratio, which is calculated as a ratio of the peak height to a noise, greater than a ratio given value and a noise greater than a noise given value, in a peak region;
step S136, determining a region having specific estimated line widths of each peak as a fitted region, and in a case of no overlapping peaks, fitting a single Gaussian peak by Levenberg-Marquardt algorithm to find specified parameters to minimize a tuning function.

7. The numerical processing method for the nucleic acid mass spectrum according to claim 3, wherein the self-weighted average method is described by using a following equation:
$$\overline{{I}_{i}} = \frac{{\sum }_{j = 1}^{n} {I}_{ij}^{2}}{{\sum }_{j = 1}^{n} \vert {I}_{ij} \vert} ,$$
wherein n is a count of the plurality of mass spectra, Īᵢ is an average intensity of mass i; Iᵢⱼ is an intensity of the mass i from a j-th mass spectrum.

8. The numerical processing method for the nucleic acid mass spectrum according to claim 6, wherein in the step S131, the expected line width determined is described by a following equation:
λₑ=L_{A}+L_{B}·M
wherein L_{A} and L_{B} are default parameters, and M is a given peak value.

9. The numerical processing method for the nucleic acid mass spectrum according to claim 6, wherein the tuning function in the step S136 is described by a following equation:
$${χ}^{2} = {Σ [ \frac{{y}_{i} - {H}_{f} \cdot exp [ - {( \frac{{M}_{f} - {m}_{i}}{{λ}_{f}} )}^{2} ]}{{σ}_{i}} ]}^{2}$$
wherein a sum is obtained by summing all {yᵢ, mᵢ} from a specified interval, H_{f} is a fitting height above the baseline corresponding to a point M_{f}; a parameter M_{f} represents a fitting mass, a parameter λ_{f} represents a fitting line width, and σᵢ is a certain parameter according to a given condition.

10. The numerical processing method for the nucleic acid mass spectrum according to claim 2, wherein the step S2 comprises:
synthesizing the plurality of mass spectra by using a self-weighted average method,
selecting an optimal mass spectrum with the most anchor peaks from the plurality of mass spectra in a case where the plurality of mass spectra have different calibration coefficients;
initializing a mass spectrum synthesized by performing the self-weighted average method on the plurality of mass spectra with the optimal mass spectrum;
summing an absolute intensity or a square intensity of a mass spectrum with an absolute intensity or a square intensity of another mass spectrum only in a case where a calibration coefficient of the mass spectrum and a calibration coefficient of the optimal spectrum meet a condition.

11. The numerical processing method for the nucleic acid mass spectrum according to claim 10, wherein in the step S13, steps of fitting a peak comprises:
step S131: determining an expected line width;
step S132: masking a region of an expected signal within an interval of NN expected line widths, NN being 4;
step S133: calculating an average of an intensity yᵢ of the single mass spectrum within a MMλm interval as an implicit baseline, wherein λm is a smallest estimated line width in the MMλm interval, MM is 80, and in a masked region of the MMλm interval, a value of the intensity yᵢ is provided by linear interpolation;
step S134: calculating an effective value of a signal-baseline operation as a noise level;
step S135: masking a point, and further masking a point, having a signal-noise ratio, which is calculated as a ratio of the peak height to a noise, greater than a ratio given value and a noise greater than a noise given value, in a peak region;
step S136, determining a region having specific estimated line widths of each peak as a fitted region, and in a case of no overlapping peaks, fitting a single Gaussian peak by Levenberg-Marquardt algorithm to find specified parameters to minimize a tuning function.

12. The numerical processing method for the nucleic acid mass spectrum according to claim 2, wherein the step S3 comprises:
performing wavelet filtering on the unitary mass spectrum of the detection point to eliminate the high-frequency noise and the baseline to obtain a filtered mass spectrum, and then performing another round of recalibration on the filtered mass spectrum and adjusting a mass-to-charge ratio value accordingly.

13. The numerical processing method for the nucleic acid mass spectrum according to claim 12, wherein in the step S13, steps of fitting a peak comprises:
step S131: determining an expected line width;
step S132: masking a region of an expected signal within an interval of NN expected line widths, NN being 4;
step S133: calculating an average of an intensity yᵢ of the single mass spectrum within a MMλm interval as an implicit baseline, wherein λm is a smallest estimated line width in the MMλm interval, MM is 80, and in a masked region of the MMλm interval, a value of the intensity yᵢ is provided by linear interpolation;
step S134: calculating an effective value of a signal-baseline operation as a noise level;
step S135: masking a point, and further masking a point, having a signal-noise ratio, which is calculated as a ratio of the peak height to a noise, greater than a ratio given value and a noise greater than a noise given value, in a peak region;
step S136, determining a region having specific estimated line widths of each peak as a fitted region, and in a case of no overlapping peaks, fitting a single Gaussian peak by Levenberg-Marquardt algorithm to find specified parameters to minimize a tuning function.

14. The numerical processing method for the nucleic acid mass spectrum according to claim 3, wherein the step S3 comprises:
performing wavelet filtering on the unitary mass spectrum of the detection point to eliminate the high-frequency noise and the baseline to obtain a filtered mass spectrum, and then performing another round of recalibration on the filtered mass spectrum and adjusting a mass-to-charge ratio value accordingly.

15. The numerical processing method for the nucleic acid mass spectrum according to claim 14, wherein the self-weighted average method is described by using a following equation:
$$\overline{{I}_{i}} = \frac{{\sum }_{j = 1}^{n} {I}_{ij}^{2}}{{\sum }_{j = 1}^{n} \vert {I}_{ij} \vert} ,$$
wherein n is a count of the plurality of mass spectra, Īᵢ is an average intensity of mass i; Iᵢⱼ is an intensity of the mass i from a j-th mass spectrum.

16. The numerical processing method for the nucleic acid mass spectrum according to claim 2, wherein the step S4 comprises:
step S41: fitting a peak of the unitary mass spectrum to obtain a fitted peak;
step S42: recording following features:
1, a height above a baseline of a center of the fitted peak, H_{f},
2, a fitting line width, λ_{f},
3, a peak offset, which is a distance between the center of the fitted peak and a center of an expected peak, δ_{f}=M_{f}−Mₑ,
4, an area A of a region between the fitted peak and the baseline within a range of 4λ_{f},
5, a signal-noise ratio, SNR=H_{f}/N(M_{f}),
6, an area variance, V=A/SNR,
7, a fitting area difference Δ, a square root of a sum of a square difference between a fitted intensity and a measured intensity.

17. The numerical processing method for the nucleic acid mass spectrum according to claim 16, wherein in the step S13, steps of fitting a peak comprises:
step S131: determining an expected line width;
step S132: masking a region of an expected signal within an interval of NN expected line widths, NN being 4;
step S133: calculating an average of an intensity yᵢ of the single mass spectrum within a MMλm interval as an implicit baseline, wherein λm is a smallest estimated line width in the MMλm interval, MM is 80, and in a masked region of the MMλm interval, a value of the intensity yᵢ is provided by linear interpolation;
step S134: calculating an effective value of a signal-baseline operation as a noise level;
step S135: masking a point, and further masking a point, having a signal-noise ratio, which is calculated as a ratio of the peak height to a noise, greater than a ratio given value and a noise greater than a noise given value, in a peak region;
step S136, determining a region having specific estimated line widths of each peak as a fitted region, and in a case of no overlapping peaks, fitting a single Gaussian peak by Levenberg-Marquardt algorithm to find specified parameters to minimize a tuning function.

18. The numerical processing method for the nucleic acid mass spectrum according to claim 3, wherein the step S4 comprises:
step S41: fitting a peak of the unitary mass spectrum to obtain a fitted peak;
step S42: recording following features:
1, a height above a baseline of a center of the fitted peak, H_{f},
2, a fitting line width, λ_{f},
3, a peak offset, which is a distance between the center of the fitted peak and a center of an expected peak, δ_{f}=M_{f}−Mₑ,
4, an area A of a region between the fitted peak and the baseline within a range of 4λ_{f},
5, a signal-noise ratio, SNR=H_{f}/N(M_{f}),
6, an area variance, V=A/SNR,
7, a fitting area difference Δ, a square root of a sum of a square difference between a fitted intensity and a measured intensity.

19. The numerical processing method for the nucleic acid mass spectrum according to claim 18, wherein the self-weighted average method is described by using a following equation:
$$\overline{{I}_{i}} = \frac{{\sum }_{j = 1}^{n} {I}_{ij}^{2}}{{\sum }_{j = 1}^{n} \vert {I}_{ij} \vert} ,$$
wherein n is a count of the plurality of mass spectra, Īᵢ is an average intensity of mass i; Iᵢⱼ is an intensity of the mass i from a j-th mass spectrum.

20. The numerical processing method for the nucleic acid mass spectrum according to claim 4, wherein the step S4 comprises:
step S41: fitting a peak of the unitary mass spectrum to obtain a fitted peak;
step S42: recording following features:
1, a height above a baseline of a center of the fitted peak, H_{f},
2, a fitting line width, λ_{f},
3, a peak offset, which is a distance between the center of the fitted peak and a center of an expected peak, δ_{f}=M_{f}−Mₑ,
4, an area A of a region between the fitted peak and the baseline within a range of 4λ_{f},
5, a signal-noise ratio, SNR=H_{f}/N(M_{f}),
6, an area variance, V=A/SNR,
7, a fitting area difference Δ, a square root of a sum of a square difference between a fitted intensity and a measured intensity.
